(19) United States
(12) Patent Application Publication
(10) Pub. No.: US 2025/0244245 A1
(43) Pub. Date: Jul. 31, 2025

(54) SYSTEM AND METHOD FOR DETERMINING THE PRESENCE OF CHROMOPHORES WITHIN AN ASSAY

(71) Applicant: AFIMACHECK INC., Toronto (CA)

(72) Inventors: Nima TABATABAEI, Toronto (CA); Derek HAYDEN, Toronto (CA)

(21) Appl. No.: 19/032,535

(22) Filed: Jan. 21, 2025

Related U.S. Application Data
(60) Provisional application No. 63/625,074, filed on Jan. 25, 2024.

(51) Int. Cl. G01N 21/71; G01N 33/53; G01N 33/543

(52) U.S. Cl. CPC G01N 21/71; G01N 33/5308; G01N 33/54388

(57) ABSTRACT

System and method for determining the presence of chromophores within an assay, the method including: directing a light source at a surface of the assay to thermally excite chromophore particles of interest; receive radiometric measurements from an opposing surface of the assay; and detecting thermal wave responses of surface or subsurface chromophore particles within the assay from the received radiometric measurements. In some cases, detecting thermal waves as a radiometric signal from one or more test lines of the assay and detecting thermal waves as a radiometric signal from a control line of the assay; and wherein detecting the thermal wave responses includes determining an intensity of the detected thermal waves from the one or more test lines and the control line, normalizing the thermal-wave response of the one or more test lines to the thermal-wave response of the control line, and outputting the normalized thermal-wave response.

### TECHNICAL FIELD

The present invention relates generally to the field of analyte detection devices, and more particularly, to a system and method for determining the presence of chromophores within an assay.

### BACKGROUND

Lateral flow assay (LFA) tests, otherwise referred to as rapid tests, are inexpensive paper-based devices for rapid and specific detection of an analyte of interest (e.g., COVID virus) in fluidic samples. LFA tests can be generally performed on-site by non-specialized users, and are generally significantly cheaper than laboratory-based alternatives. For example, LFA tests allow for medical diagnostic testing at or near the time and place of patient care; in contrast to testing that is wholly or mostly confined to medical laboratories. LFA devices are particularly advantageous due to, for example, low-cost commercial viability, rapid identification of disease or biochemical conditions, ease of use with minimal user training, device portability at a physician's office or at a patient's side, and device robustness and disposability that eliminates contamination or safety hazards. Due to these benefits, LFA tests are typically used in a wide range of applications, including agriculture, water safety, and point-of-care medical testing.

Despite the advantages of LFA tests, they typically suffer from limitations, such as having far inferior limits of detection and sensitivity when compared to standard laboratory tests. Moreover, LFAs results are normally binary, a positive or negative response indicating presence or absence of an analyte above a certain concentration. While this response is useful in many scenarios, there are many other applications which would benefit from reliable quantification of analyte concentration. For example, in roadside testing for levels of tetrahydrocannabinol (THC) in oral fluids, the limit-of-detection of common types of LFAs are between 25 ng/ml to 40 ng/ml; while a typical recommended legal limit for driving is between 0 to 2 ng/ml. As such, determinations of THC intoxication at a roadside requires use of more invasive, time-consuming, and costly tests such as a blood test administered by a specialized professional.

### SUMMARY

In an aspect, there is provided a system for determining the presence of chromophores within an assay, the system comprising: one or more intensity-modulated thermal excitation sources directed at a surface of the assay to thermally excite chromophore particles of interest; a thermal capture device oriented toward an opposing surface of the assay to receive radiometric measurements; and a computing device, in communication with the thermal capture device, to receive the radiometric measurements and determine thermal wave responses of surface or subsurface chromophore particles within the assay from the received radiometric measurements.

In a particular case of the system, the assay is a microfluidic assay.

In another case of the system, the backing of the microfluidic assay is substantially transparent to excitation from the one or more intensity-modulated thermal excitation sources, emission, or both.

In yet another case of the system, the thermal capture device is a single element infrared sensor.

In yet another case of the system, the infrared capture device comprises an array of infrared sensors.

In yet another case of the system, the one or more intensity-modulated thermal excitation sources comprise a light source.

In yet another case of the system, the light source comprises a light emitting diode or array of light emitting diodes.

In yet another case of the system, the light source emits light at a plurality of selected wavelengths to selectively excite a plurality of chromophores of interest.

In yet another case of the system, the computing device further determines a depth profilometry by changing an optical modulation frequency.

In yet another case of the system, the chromophore particles are gold nanoparticles (GNPs).

In yet another case of the system, multiple chromophore particles are selected to selectively absorb the energy from the thermal excitation source at different wavelengths such that thermal waves are produced by a plurality of wavelengths at one or more modulation frequencies.

In another aspect, there is provided a method for determining the presence of chromophores within an assay, the method comprising: directing an intensity-modulated thermal excitation source at a surface of the assay to thermally excite chromophore particles of interest; receive radiometric measurements from an opposing surface of the assay; and detecting thermal wave responses of surface or subsurface chromophore particles within the assay from the received radiometric measurements.

In a particular case of the method, receiving the radiometric measurements comprises detecting thermal waves as a radiometric signal from one or more test lines of the assay and detecting thermal waves as a radiometric signal from a control line of the assay; and wherein detecting the thermal wave responses comprises determining an intensity of the detected thermal waves from the one or more test lines and the control line, normalizing the thermal-wave response of the one or more test lines to the thermal-wave response of the control line, and outputting the normalized thermal-wave response.

In another case of the method, the method further comprising selecting multiple chromophore particles to be thermally excited such that emission of thermal waves is conducted at a plurality of wavelengths.

In yet another case of the method, the method further comprising selecting multiple chromophore particles to selectively absorb energy from the one or more thermal excitation sources at different wavelengths such that thermal waves are produced by a plurality of wavelengths at one or more modulation frequencies.

These and other embodiments are contemplated and described herein. It will be appreciated that the foregoing summary sets out representative aspects of various embodiments to assist skilled readers in understanding the following detailed description.

### DESCRIPTION OF THE DRAWINGS

A greater understanding of the embodiments will be had with reference to the Figures, in which:

FIG. 1 shows a schematic diagram of a system for detection of analytes in a lateral flow assay (LFA), in accordance with an embodiment;

FIG. 2 shows a rendering of an example physical implementation of the system of FIG. 1;

FIG. 3 shows a schematic diagram of an example implementation of the system of FIG. 1;

FIG. 4 shows a flowchart of a method for detection of analytes in a lateral flow immunoassay (LFA), in accordance with an embodiment;

FIG. 5A is a chart showing interpolated and resampled time series of control and test line responses for example experiments;

FIG. 5B is a chart showing control and test line responses transformed to the frequency domain for the example experiments; and

FIG. 6 is a chart showing a normalized thermal wave response for the example experiments.

In the drawings, embodiments of the invention are illustrated by way of example. It is to be expressly understood that the description and drawings are only for the purpose of illustration and as an aid to understanding, and are not intended as a definition of the limits of the invention.

### DETAILED DESCRIPTION

For simplicity and clarity of illustration, where considered appropriate, reference numerals may be repeated among the Figures to indicate corresponding or analogous elements. In addition, numerous specific details are set forth in order to provide a thorough understanding of the embodiments described herein. However, it will be understood by those of ordinary skill in the art that the embodiments described herein may be practised without these specific details. In other instances, well-known methods, procedures and components have not been described in detail so as not to obscure the embodiments described herein. Also, the description is not to be considered as limiting the scope of the embodiments described herein.

Various terms used throughout the present description may be read and understood as follows, unless the context indicates otherwise: “or” as used throughout is inclusive, as though written “and/or”; singular articles and pronouns as used throughout include their plural forms, and vice versa; similarly, gendered pronouns include their counterpart pronouns so that pronouns should not be understood as limiting anything described herein to use, implementation, performance, etc. by a single gender. Further definitions for terms may be set out herein; these may apply to prior and subsequent instances of those terms, as will be understood from a reading of the present description.

Any module, unit, component, server, computer, terminal or device exemplified herein that executes instructions may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the device or accessible or connectable thereto. Further, unless the context clearly indicates otherwise, any processor or controller set out herein may be implemented as a singular processor or as a plurality of processors. The plurality of processors may be arrayed or distributed, and any processing function referred to herein may be carried out by one or by a plurality of processors, even though a single processor may be exemplified. Any method, application or module herein described may be implemented using computer readable/executable instructions that may be stored or otherwise held by such computer readable media and executed by the one or more processors.

The present disclosure relates generally to the field of analyte detection devices, and more particularly, to a system and method for determining the presence of chromophores within an assay.

There are a number of approaches aimed at retaining the universal use and inexpensive attributes of the lateral flow assay (LFA) tests while increasing their sensitivity and limit-of-detection. A particular approach can use LFAs that contain fluorescent polymeric beads; allowing for fluorescent interrogation of the LFA to enhance the limit of detection. However, LFAs are generally not manufactured using fluorescent beads as they require specialized equipment to interpret, have issues with stability and are substantially more expensive to develop relative non-fluorescent assays (e.g., gold nanoparticle (GNP) assays).

Another approach that requires no change in LFA manufacturing is thermal contrast analysis. In such an approach, a focused laser light is used to excite gold nanoparticles that are located in a standard LFA. The resulting thermal radiation is measured using an infrared camera. This type of sensing can increase the sensitivity and limit of detection of LFAs, while retaining the low-cost and rapid attributes of LFAs. However, such approaches are based on measuring the temperature which makes the method/analysis prone to errors caused by changes in ambient temperature. Moreover, thermal contrast methods normally utilize a thermal camera, which can be prohibitively expensive for end-users; especially considering LFAs are highly valued for being inexpensive.

Embodiments of the present disclosure integrate a sensing paradigm of photothermal radiometry (PTR) into a system that utilizes inexpensive single-element far-infrared thermal sensors. Advantageously, embodiments of the present disclosure significantly decrease the device cost and size while offering enhanced sensitivity and limit of detection of LFA tests, allowing for significant enhancement of the detection performance of commercially-available rapid tests.

Generally, an LFA test is performed by depositing a controlled volume of fluidic specimen onto a sample pad. This fluid is then pulled by capillary action through a membrane to a conjugate release pad, which contains antibodies specific to a type of analyte being tested. In some cases, the target analyte blocks binding sites in test line(s), allowing the antibodies to pass the test line and instead bind to a control line. In such cases, spiking the LFA with specimens containing a concentration of analyte above the detection limit of the test (i.e., a positive outcome) yields a test strip with a non-visible, or minimally-visible, test line(s) and a visible control line.

LFAs can use GNPs as visible indicators for both test and control lines. These GNPs can be used to selectively reflect and absorb ambient lighting, leading to colorimetric signals (normally dark-purplish color) at the test and control lines. Embodiments of the present invention advantageously use photothermal radiometry (PTR) approaches that take advantage of selective absorption spectra of GNPs; such that there are specific wavelengths at which light is absorbed very well by the GNPs and absorbed very weakly by a surrounding nitrocellulose paper. Accordingly, the PTR sensing approach normally excites LFAs with a laser or LED excitation at specific wavelength in order to induce amplified thermal wave responses. Such responses can simultaneously be measured with single element infrared (IR) sensors and then quantified to yield signals that are proportional to the extent of accumulation of GNP-analyte pairs at the LFA lines. To make PTR sensing insensitive to varying ambient temperatures, or other sources of ambient thermal noise, the laser excitation can be intensity modulated at a specific frequency (e.g., 1 Hz) or a collection of frequencies (e.g., frequency chirp of 0.1-10 Hz or binary/Barker phase coded pattern with 1 Hz carrier frequency) to create thermal wave fields within the thickness of the LFAs. Demodulation of acquired IR signals in accordance with the intensity modulation method (e.g., at the specific frequency) enables removal of environmental thermal effects to enable very sensitive detection and quantification of light absorption signatures of the GNP-analyte complexes.

While the present disclosure generally makes reference to LFA tests, it is understood that the system and methods described herein can be applied to any suitable microfluidic assay.

Advantageously, embodiments of the present disclosure can use inexpensive single-element IR sensors to produce results that are comparable to those produced by significantly more expensive laser and thermal-camera based systems; for example, using an inexpensive single-element far-infrared sensor and a 1.6 W (electrical input power) LED diode. In such arrangements, the reduced optical excitation power and sensor sensitivity is overcome by sandwiching the assay between excitation source and detector; i.e., between and proximate to the thermal excitation source and sensors. Minimizing the distance between the components and the assay region of interest maximizes efficacy of the provided optical power of LED/laser and is optimal for collecting the ensuing radiometric emissions.

In various embodiments, parametric optimization can be used, whereby one of the design variables (e.g., light beam size) can be modified and tests can be performed on the control LFAs. Configuration performance can be compared with previous configurations and further optimizations can be performed on the design parameters. The design of the assay can also be modified to include materials, particularly the LFA plastic backing, which are at least partially transparent to incident modulating light, allowing for unimpeded light incident on the GNPs. As the assay backing is typically plastic, there are several choices with varying optical properties. These improvements increase light incident on the chromophores of interest and/or infrared sensors thereby increasing overall signal and improving level of detection.

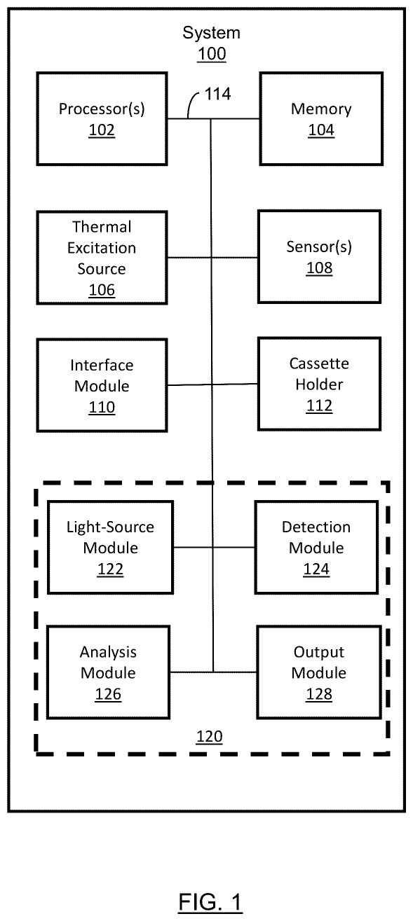

FIG. 1 illustrates a system for determining the presence of chromophores within an assay 100, in accordance with an embodiment. The system 100 comprises one or more processors 102, a data memory 104, a thermal excitation source 106, one or more sensors 108, an interface module 110, a cassette holder 112, and a local bus 114 that enables the one or more processors 102 to communicate with the other components. The one or more processors 102 can include one or more central processing units, one or more graphical processing units, microprocessors (such as an Arduino™), dedicated hardware, or other integrated processing circuits. The interface module 110 enables input to be provided; for example, directly via a user input device, or communicated, for example, via an external device or system. The interface module 110 also enables output to be provided; for example, directly via a user display, or indirectly, for example, communicated over a network with other systems or computing devices (for example, over a local 11 area network or over the Internet). The data memory 104 provides data storage to the processing unit 102; such as, computer-executable instructions for implementing the methods described herein, as well as any derivative or related data. In other embodiments, any operating system, programs, or instructions can be executed in hardware, specialized microprocessors, logic arrays, or the like. While FIG. 1 illustrates a system implemented on a single computing device, it is understood that the processing, or any of the functions undertaken by the system 100, can be distributed over multiple computing devices; for example, in a cloud or distributed computing environment.

In an embodiment, the one or more processors 102 can be configured to execute a number of conceptual modules 120; for example, an excitation-source module 122, a detection module 124, an analysis module 126, and an output module 128. In further cases, functions of the above modules can be combined or executed on other modules. In some cases, functions of the above modules can be executed on remote computing devices, such as centralized servers and cloud computing resources communicating over the network.

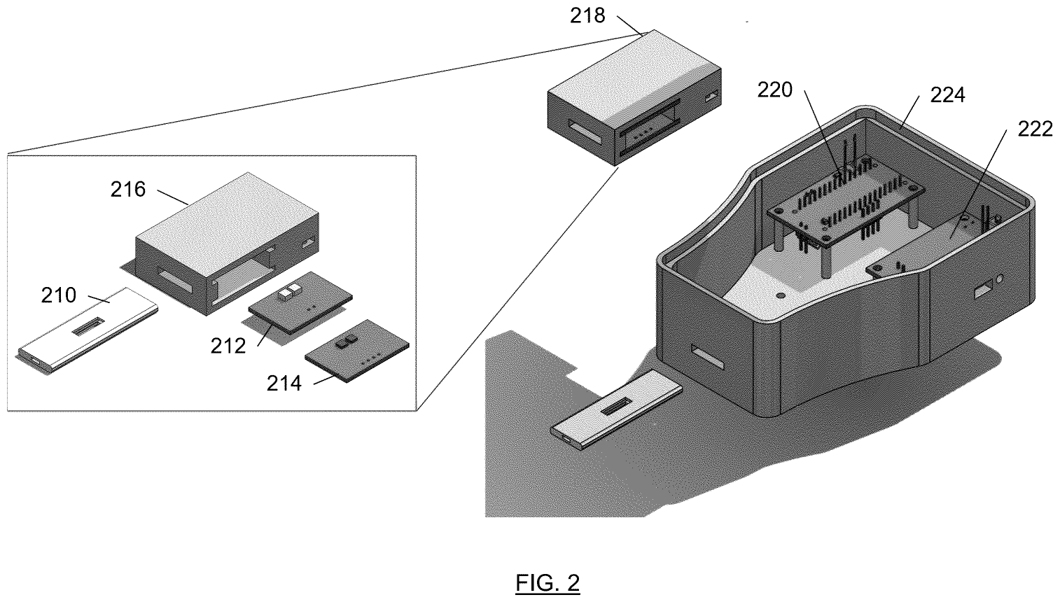

FIG. 2 illustrates an example physical rendering of an implementation of the system 100. In this example, the one or more processors 102 and the data memory 104 are located on a first printed circuit board (PCB) 220, the thermal excitation source 106 is located on a second PCB 212, the one or more sensors 108 are located on a third PCB 214, and the cassette holder 112 is embodied by item 210. An LFA cassette 210, with viewing ports on both sides, allows for transmissive interrogation. The second PCB 212 contains two LEDs, spaced to be coincident with the test and control lines of the particular LFA being interrogated. The third PCB contains two single element sensors, spaced the same as the second PCB. A sensing housing 216 holds the LFA cassette 210 and the LED and sensor components in proper orientation. Item 218 shows the sensing housing 216 assembled with all the second PCB 212 and the third PCB 214 located therein. The assembled sensing housing 216 modularly fits into the handheld device to allow for LFAs of different spacing to be interrogated using the same device. The first PCB 220 contains, for example, a microcontroller to modulate the LED and receive signals from the sensors. The microcontroller can also process the sensor signals and transmit the raw signals and processed data to other devices, such as wirelessly to cloud storage or to a personal device. A fourth PCB controls external power in order to charge an onboard battery as required. A device housing 224 contains the aforementioned components in order to shield them as required.

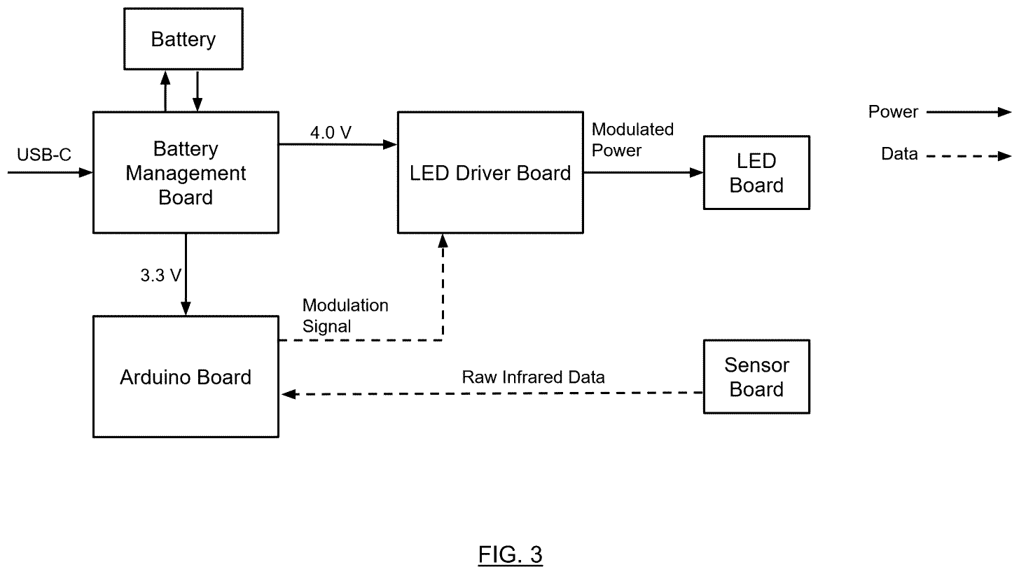

FIG. 3 illustrates a schematic diagram of an example of the system 100. In these examples, the one or more processors 102 and data memory 104 comprise an Arduino™ microcontroller implementation. The one or more sensors 108 comprise a far-infrared single-element sensor (e.g., the Melexis MLX90632 non-contact infrared temperature sensor). In these examples, the cassette holder 112 holds an LFA for analysis. While the thermal excitation source 106 is generally described herein using a light-emitting-diode (LED), any suitable modulated thermal excitation source can be used; for example, a laser, a generator of an alternating magnetic field, a generator of a voltage potential, or the like. Generally, as the thermal excitation source 106 is brought closer to the LFA, a lower power excitation-source can be used.

In the example of FIG. 3, a battery management circuit board regulates the external power, routing it to an LED driver circuit board, microcontroller board, or battery if the battery needs charging. When the battery is full and there is no external power source, the battery management board regulates the power from the onboard battery. The LED driver circuit board regulates and modulates the power sent to the LEDs based on the modulation signal sent by the microcontroller. The microcontroller circuit board houses the Arduino™ microcontroller which sends a modulation signal to the LED driver board, and receives the raw signal from the single-element sensors on a sensor circuit board.

The system 100 uses an intensity-modulated thermal excitation sources to excite LFA GNPs while registering their thermal-wave responses with single-element infrared sensors. The system 100 can perform frequency-domain analysis of the radiometric signals to enable sensitive detection and quantification of photothermal radiometric responses of the LFAs; which translates to significant enhancement of the LFA nominal detection threshold. Results of example experiments show that, through calibration, the end user device can coarsely quantify the concentration of analyte used for spiking the LFA.

Intensity modulated excitation, as described in the present disclosure, can take one of many forms; such as single-frequency intensity modulation, multi-frequency modulation, frequency scan modulation, or binary or Barker phase coded modulation. Accordingly, thermal wave detection from the radiometric signal can involve appropriate signal processing; for example, lock-in demodulation or matched filtering (e.g., cross-correlation).

Initial calibration of this device for use with an assay is performed by first preparing control positive solutions at a variety of concentrations through a method of serial dilution. These solutions are used to prepare several assays at each concentration. These assays are then measured using the device, and the known concentrations are paired with the normalized responses. These data pairs are then fit to a logistic curve, producing a calibration curve. Subsequent tests with unknown concentrations can then use this curve to predict the concentration of analyte with the measurement produced by the device. The calibration can be verified and improved upon as additional assays are tested using the device when paired with subsequent laboratory results at a known sensitivity. Alternately, calibration can be done via machine and deep learning methods. In such approaches, the machine or deep learning model is trained by historic device data (e.g., raw sensor data or normalized intensity data) labelled with corresponding true analyte concentrations. The trained model can then predict the concentration of analyte in fluidic samples.

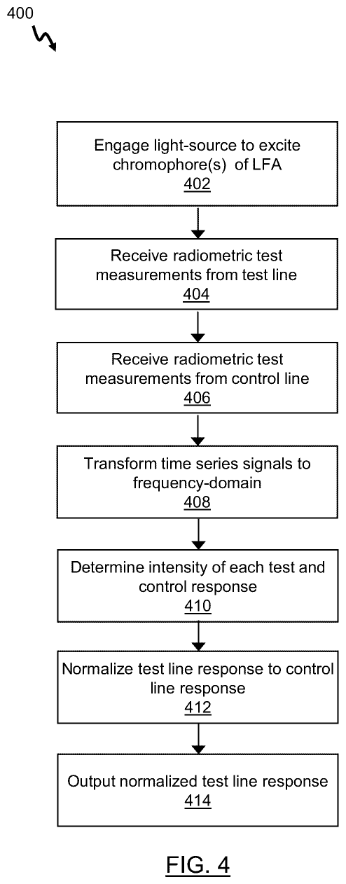

FIG. 4 illustrates an embodiment of a method 400 for determining the presence of chromophores within an assay. At block 402, the excitation-source module 122 engages the thermal excitation source 106 to thermally excite chromophore(s) (for example, gold nanoparticles (GNPs)) of an LFA device in the cassette holder 112. In many cases, the wavelength for the thermal excitation source 106 can be tuned to a particular energy absorption band (e.g., light absorption band) of a chromophore of interest. Further, by varying the wavelength, it is possible to selectively excite several chromophores which, for example, could be used to detect or quantify multiple analytes within a single LFA. The varying of wavelengths may be accomplished with a single or a plurality of thermal-excitation assemblies (e.g., one or more LEDs) using pulses of multiple frequency excitations to study the depth of the LFA at once.

Energy generated by the thermal excitation source 106 is selectively absorbed by the chromophore(s) of interest within the LFA device, generating thermal waves that are transmitted to the surface of the LFA device through thermal radiation and heat conduction. In this way, conducted thermal waves reach the surface and then radiometrically contribute to the sensor signal. In addition, the energy (e.g., light) absorbing GNPs create radiometric emissions that can transmit through nitrocellulose paper and reach the sensor.

At block 404, the detection module 124 receives radiometric test measurements from the single-element IR sensor 108, which repeatedly detects radiometric signals from the chromophore(s) of interest of one or more test lines. At block 406, the detection module 124 receives radiometric control measurements from the single-element IR sensor 108, which repeatedly detects radiometric signals from the chromophore(s) of interest of one or more control lines. In this way, during operation, datasets can be collected separately for the test and control lines of the LFA. Both datasets contain a time series of thermal-wave radiometric measurements from the single-element sensor.

Generally, such time series measurements do not have to be perfectly spaced samples in time; such that the time series can be interpolated and resampled at regular time intervals to enable more accurate frequency-domain analysis of the signals. At block 408, in some cases, the analysis module 126 transforms the time series signals to the frequency-domain by, for example, applying a Fast Fourier Transform (FFT) on the resampled time-series. It is appreciated that in other cases, the time series signals may not be required to be transformed into the frequency-domain.

At block 410, the analysis module 126 determines the intensity of each thermal-wave response, for the test measurements and the control measurements. In some cases, this intensity can be determined with a comparison of the thermal-wave response at the modulation frequency (e.g., 1 Hz) of the thermal excitation source 106 to a respective noise floor. In some cases, the noise floor can be selected to be a signal response from nitrocellulose paper where no or few chromophores are present. The noise floor represents a minimum intensity that a signal needs to have in order to be determinable when compared to the inherent noise of the other elements of the system.

At block 412, the analysis module 126 normalizes the thermal-wave response intensity of the test line to the thermal-wave response intensity of the control line (e.g., by division of amplitudes or subtraction of phases). Any normalization approach can be used, for example, dividing the response of the test line by the response of the control line, or subtraction of the signal phase of the test line from the signal phase of the control line. The normalized thermal-wave response, which can be referred to as an amplitude metric, is strongly correlated to the concentration of analyte in the fluid under test in the corresponding LFA.

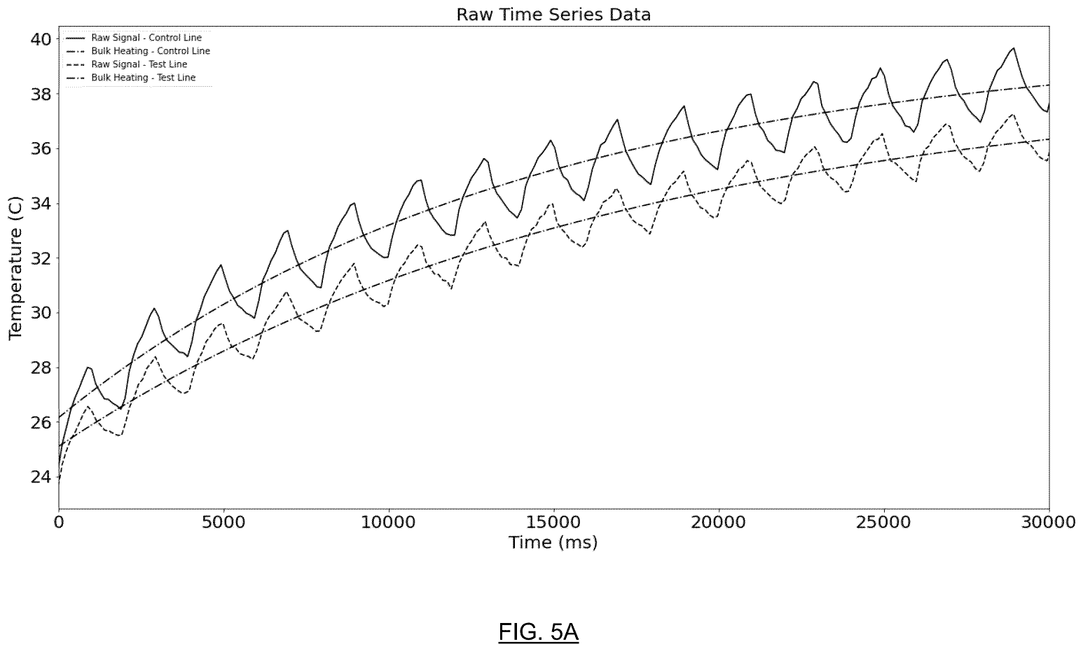

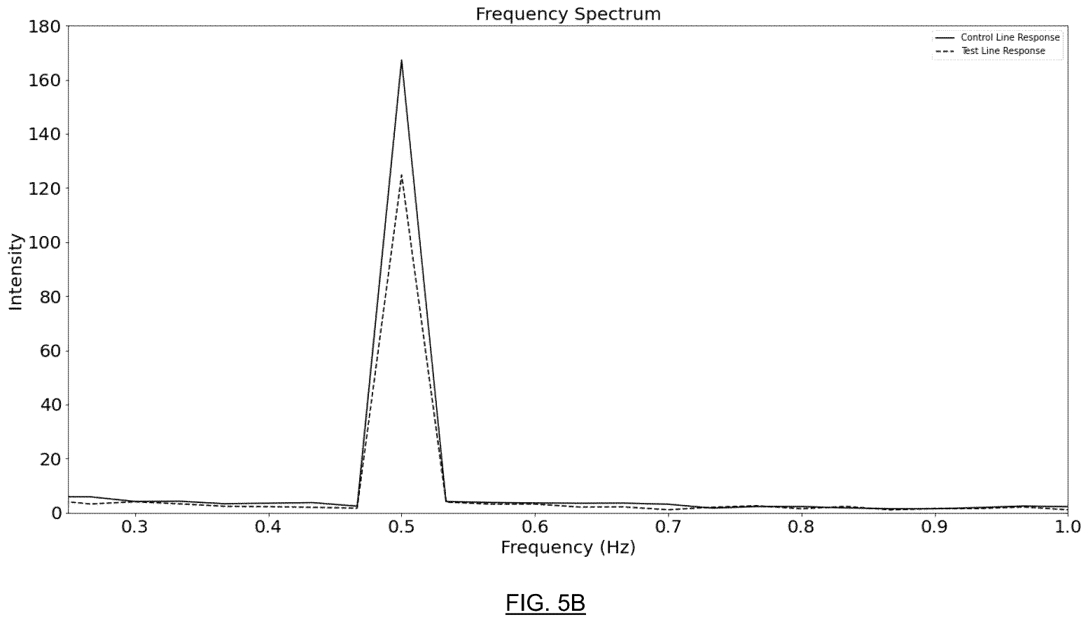

FIGS. 5A and 5B show charts of representative interpolated and resampled time series data, and the corresponding frequency-domain spectra, for an LFA's control and test lines in an example experiment. As shown, the thermal-wave response is stronger for the control line in both time and frequency domains compared to those of the test line. FIG. 5A shows interpolated and resamples time series of control and test line responses, and FIG. 5B shows control and test line responses transformed to the frequency domain. In FIG. 5A, the bulk heating, or non-periodic, portion of the signal, is outlined. This received signal is clearly discernible from the ambient noise of the system, which demonstrates the optimization of the system 100 before any signal processing occurs. The example experiment was performed at a modulation frequency of 0.5 Hz, and FIG. 5B illustrates an overwhelmingly positive signal-to-noise ratio achievable by the design of the system 100.

At block 414, the output module 128 outputs the normalized thermal-wave response to the memory 104 and/or the interface module 110 for use in the detection and quantification of analytes. In some cases, the output module 128 can interface with other devise to provide the output to such devices, for example, over a network; such as with a personal computing device or a cloud storage.

In some cases, the calibration curve described herein can have two axes: (1) “concentration” and (2) “normalized response”. When an unknown LFA is tested, the outputted normalized response determined at block 412 can be compared to the calibration curve, and where the response intersects with the calibration curve provides an estimation of the concentration. In further cases, the normalized thermal-wave response determined at block 412 can be inputted into a machine learning model to determine as estimation of the concentration; where such model is trained with a training dataset comprising thermal-wave responses and associated detections and quantifications of analytes.

In the example experiments, to test the performance of the system 100, twenty LFAs were spiked with five known concentrations of THC (i.e., four spiked LFAs at each of the five concentrations). Spiking solutions were prepared by mixing a known volume of Delta-9 THC stock solution with known volume of artificial saliva. Measured quantities of this solution were then serially diluted to obtain THC concentrations of 0 ng/ml, 2.5 ng/ml, 5 ng/ml, 10 ng/ml, 25 ng/ml. Twenty LFAs, each with a cutoff concentration of 25 ng/ml, were then spiked using 200 ul of their respective solution and labelled accordingly.

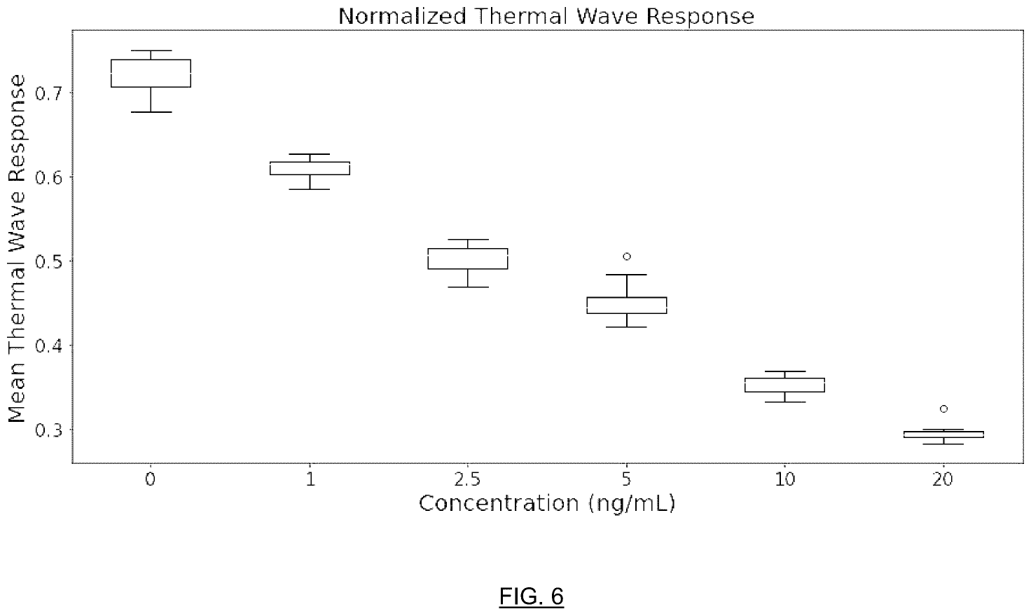

FIGS. 5A and 5B depict an amplitude metric of mean±STD for data collected from the twenty LFAs in the example experiments. FIGS. 5A and 5B depict results of 5 repetitions per LFA (N=20 per concentration) using a custom cassette. FIG. 5A shows a chart of a control line versus normalized test line response. Given the design of the LFAs, as THC concentration increases, the test line fades, leading to a smaller amplitude metric signal. Results depicted in FIG. 6 show that for THC concentrations greater than 2.5 ng/mL, the amplitude metric of test line is significantly smaller than that of the LFA's control line. In this example, the limit of detection was measured at 5 ng/ml.

FIG. 6 shows an example experiment that contained THC metabolite LFAs with a nominal cutoff concentration/limit of detection of 15 ng/ml. Four LFAs were prepared at each of the concentrations, as shown on the X axis. The boxplot demonstrates the interquartile ranges of the responses received by the device. These example results demonstrate the significant improvement in the limit of detection of the LFA as a result of interrogation by the device, as well as illustrate the quantitative capabilities made possible by the present embodiments.

FIG. 6 shows the collected data from all THC concentrations tested. Qualitative assessment shows correlation of end-user output with THC concentration. To assess the data quantitatively, one-way ANOVA analysis of means was performed which suggested presence of statistical significance (p<0.05). As such, a Tukey HSD post-hoc pairwise comparison was conducted to identify which pairs of means were significantly different. As depicted in FIG. 6, results suggest that the system 100, in the form of an end-user device, can be reliably resolved the THC concentration of the sample into one of four categories (<5 ng/ml, 5-10 ng/ml, 10-25 ng/mL, >25 ng/ml).

FIG. 6 shows two substantial advantages of the present system 100: (1) the system 100 has an enhanced detection limit from a nominal LFA threshold of 25 ng/ml to 1 ng/ml; and (2) the system 100 enables semi-quantitative detection of THC into 4 concentration categories (most of which are below nominal detection threshold). For point (2), this is a substantial advantage over conventional approaches that use binary categorization of the LFA (i.e., <25 ng/ml OR>25 ng/ml).

A further substantial advantage of the present system 100 is the potential low-cost nature of the components. The system 100 is considerably less expensive than even the cheapest thermal camera-based LFA reader, which currently is approximately USD $350, while the cost of an example implementation of the system 100 is approximately USD $35: two LEDs and related electronics—$5, 2 IR Sensors—$15, Arduino-based microprocessor—$12, and housing $3.

It is understood that the system 100 can be implemented with other components than those provided in the example experiments. For example, for increasing sensitivity, one or more thermal excitation sources 106 of one or more different wavelengths can be used, particularly wavelengths at which GNPs are more absorptive. In this way, multiple chromophore particles are selected to selectively absorb the energy from the thermal excitation sources of different wavelengths such that thermal waves are produced by a plurality of wavelengths at one or more modulation frequencies. In another example, a more sensitive sensor can be used to increase the data quality, and therefore, potentially increase overall sensitivity of the device. Further, embodiments of the present disclosure can also be used in ‘transmission mode’ where the backing of the LFA is transparent to light that is being used to excite the chromophores. The light that is used to excite the chromophores can be located adjacent to the transparent backing, and the sensor on the other side of the LFA. This transmissive mode allows for both lighting and sensing elements to be as close as possible to the LFA, which can greatly increase the signal-to-noise ratio (SNR).

Advantageously, the present embodiments provide a low-cost system that is useable by an end-user without the need for laboratory testing; such that there is substantial enhancement of the detection performance of rapid tests using LFAs. Alternatively, the present embodiment can be used in a laboratory setting as an alternative to more costly and time-consuming laboratory methods such as enzyme linked immunoassays.

It will be appreciated that the presently disclosed system may be applied to other devices beyond LFA. Whilst the application to LFA is based on the chromophore(s) of interest, it may be applied to other targets/analytes of interest in other applications. For example, the presently disclosed system may be applied to microfluidic chips, with suitable adjustments to the wavelengths of the thermal excitation source and the thermal capture device as appropriate for targets or analytes of interest in the microfluidic chips. As an additional example, the presently disclosed system may be applied to vertical flow assays wherein the arrangement of sample pad, conjugate pad, and results are in a vertical configuration.

It will be appreciated this technology can be applied to applications in which the analyte detection is not antibody-based. For example, LFAs can be based on aptamers or designed with the ability to detect specific nucleic acids that bind selected oligonucleotides conjugated with gold nanoparticles. Additionally specific minerals can be detected with properly selected ionophores conjugated to gold nanoparticles. These various known applications of LFAs are compatible with and benefit from the presently disclosed invention.

The embodiments described herein improve the sensitivity and performance of commercially available nanoparticle-based assays, but also provide benefits to non-nanoparticle-based assays once such assays are optimized for use with the system 100. Parameters such as nanoparticle size (e.g. 10 nm, 40 nm, 150 nm), nanoparticle shape (e.g. Sphere, shell, cylinder, irregular), chromophore starting distribution, binding distributions within the assay, and binding affinities can be selectively modified enhance the level of detection and/or sensitivity at the detection threshold of interest. Additionally, the composition of the chromophore particles can also be selected from a wide range of material with strong photothermal response such as gold, silver, graphene oxide, latex color beads, and engineered nanomaterials to optimize assay performance within the system.

The present embodiments provide substantial advantages over conventional colorimetric approaches that use the reflective capability of the GNPs to determine a response. For such approaches, as the assay gets more positive, the test line gets darker. In an example, the GNPs on the line absorb some of the wavelengths of light (green) and reflect others (red/purple), while the surrounding paper is white, so it always reflects all the wavelengths. At, for example, very low concentrations, there are few GNPs on the test line, and therefore, a very small amount of those wavelengths are absorbed. The surrounding paper however is still reflecting all the incoming light. This means that using a colorimetric approach at low concentrations results in attempting to look for a very small change in the light signature among a huge amount of reflective noise.

In contrast, embodiments of the present disclosure use reflectivity of the surrounding paper advantageously. Because so little light is absorbed by the paper, very little heat is produced when the LED is on. However, as soon as there are some GNPs on the line, the GNPs begin to release a relatively large amount of heat. Taking advantage of this fact, the present approaches are able to produce a relatively strong signal among a small amount of noise.

In an example where the thermal excitation source is a light source, due to not looking for very small changes in the light signature among a huge amount of reflective noise (which is necessitated in conventional approaches), the present embodiments can advantageously use low powered light systems, and cheaper and/or noisier sensors. Additionally, the present embodiments can operate well collecting a relatively small amount of data, which means less processing power is required. These factors mean that the overall cost of the system is substantially decreased compared to conventional systems.

Further advantageously, the present embodiments can employ the light source as the thermal excitation source at a surface plasmonic absorption peak of GNPS to get highly amplified signals; whereas conventional approaches that use colorimetric reading normally rely on ambient lighting.

Further advantageously, the present embodiments provide improvements to the LFA, for example: (1) LFAs with plastic backings that are not opaque to excitation or radiometric emission, or both; and (2) LFAs in which the contrast of test line(s) and control line change in an opposite way to enhance detection sensitivity.

The present embodiments generally allow for several different methods of LFA interrogation, each of which have different benefits depending on the application. Single frequency modulation is generally simplest, modulating the driving thermal excitation source at a single frequency and demodulating the response amplitude and phase at that same frequency. A matched-filtering approach modulates the thermal excitation source intensity at a collection of frequencies (e.g., a frequency scan/chirp or binary/barker coded modulation) and calculates the response via cross-correlation. In other cases, response predictions can be achieved via machine learning models or deep learning models.

While the present disclosure generally describes the present embodiments applied to LFAs, it is understood that the present embodiments can likewise be applied to microfluidic chips that operate in a similar way to LFAs.

While illustrative embodiments have been described above by way of example, it will be appreciated that various changes and modifications may be made without departing from the scope of the invention, which is defined by the following claims.

1. A system for determining the presence of chromophores within an assay, the system comprising:
one or more intensity-modulated thermal excitation sources directed at a surface of the assay to thermally excite chromophore particles of interest;
a thermal capture device oriented toward an opposing surface of the assay to receive radiometric measurements; and
a computing device, in communication with the thermal capture device, to receive the radiometric measurements and determine thermal wave responses of surface or subsurface chromophore particles within the assay from the received radiometric measurements.

2. The system of claim 1, wherein the assay is a microfluidic assay.

3. The system of claim 2, wherein the backing of the microfluidic assay is substantially transparent to excitation from the one or more intensity-modulated thermal excitation sources, emission, or both.

4. The system of claim 1, wherein the thermal capture device is a single element infrared sensor.

5. The system of claim 1, wherein the infrared capture device comprises an array of infrared sensors.

6. The system of claim 1, wherein the one or more intensity-modulated thermal excitation sources comprise a light source.

7. The system of claim 6, wherein the light source comprises a light emitting diode or array of light emitting diodes.

8. The system of claim 6, wherein the light source emits light at a plurality of selected wavelengths to selectively excite a plurality of chromophores of interest.

9. The system of claim 1, wherein the computing device further determines a depth profilometry by changing an optical modulation frequency.

10. The system of claim 1, wherein the chromophore particles are gold nanoparticles (GNPs).

11. The system of claim 1, wherein multiple chromophore particles are selected to selectively absorb the energy from the thermal excitation source at different wavelengths such that thermal waves are produced by a plurality of wavelengths at one or more modulation frequencies.

12. A method for determining the presence of chromophores within an assay, the method comprising:
directing an intensity-modulated thermal excitation source at a surface of the assay to thermally excite chromophore particles of interest;
receive radiometric measurements from an opposing surface of the assay; and
detecting thermal wave responses of surface or subsurface chromophore particles within the assay from the received radiometric measurements.

13. The method of claim 12, wherein receiving the radiometric measurements comprises detecting thermal waves as a radiometric signal from one or more test lines of the assay and detecting thermal waves as a radiometric signal from a control line of the assay; and wherein detecting the thermal wave responses comprises determining an intensity of the detected thermal waves from the one or more test lines and the control line, normalizing the thermal-wave response of the one or more test lines to the thermal-wave response of the control line, and outputting the normalized thermal-wave response.

14. The method of claim 12, further comprising selecting multiple chromophore particles to be thermally excited such that emission of thermal waves is conducted at a plurality of wavelengths.

15. The method of claim 12, further comprising selecting multiple chromophore particles to selectively absorb energy from the one or more thermal excitation sources at different wavelengths such that thermal waves are produced by a plurality of wavelengths at one or more modulation frequencies.
